(19) United States
(12) Patent Application Publication
(10) Pub. No.: US 2025/0228750 A1
(43) Pub. Date: Jul. 17, 2025

(54) POWDER FOR USE IN TREATING TOOTH SURFACES

(71) Applicant: FERTON HOLDING S.A., Delémont (CH)

(72) Inventors: Marcel DONNET, Saint Jean de Gonville (FR); Simone GATTI, Chavannes-près-Renens (CH); Alizée DARONNE, Saxel (FR)

(21) Appl. No.: 18/854,396

(22) PCT Filed: Apr. 3, 2023
(86) PCT No.: PCT/EP2023/058703

§ 371 Date: Oct. 4, 2024

(30) Foreign Application Priority Data
Apr. 5, 2022 (EP) 22166722.3

(51) Int. Cl. A61K 6/20; A61K 6/60; A61K 6/70

(52) U.S. Cl. CPC A61K 6/20; A61K 6/60; A61K 6/70

(57) ABSTRACT

The invention relates to a powder for use in treating tooth surfaces with a powder jet device, wherein the powder comprises at least one organic compound, wherein the at least one organic compound has a melting point <165° C. and a solubility in water at 25° C. of <10 g/l.

The invention relates to a powder for use in treating tooth surfaces with a powder jet device. The invention further relates to the use of the powder and a coating on tooth surfaces obtainable by applying the powder.

Professional dental prophylaxis is a maintenance treatment comprising powder jet cleaning or air polishing and aims to remove dental plaque and calculus. Such powder jet cleaning is particularly effective as it allows to reach and clean all tooth surfaces and the interspaces between them as well as implants, brackets and appliances. The professional dental prophylaxis is an important treatment that helps to maintain oral health since it removes the biofilm and calculus that a patient cannot remove during his daily home dental care.

In the powder jet cleaning process, a powder jet device is used, wherein a powder is sprayed with a gaseous carrier medium, usually air, onto tooth surfaces, which allows an efficient cleaning of the teeth. Additionally or as an alternative to a gaseous carrier medium, a liquid carrier medium, for example water may be used. Powder jet cleaning is performed with a powder jet device and it is particularly effective because it does not require repetitive movements nor different stages. Further, it is faster than other cleaning methods and it needs relatively low training to be learned correctly.

The clean teeth obtained after powder jet cleaning represent an opportunity for enhancing effects of tooth fluoridization or remineralization due to the facilitated and direct contact of the product to the tooth surface. However, additional chemical treatments require generally longer contact time to allow the chemical reactions to take place. Among these additional chemical treatments, examples are tooth fluoridization to improve the enamel acid resistance, tooth remineralization to repair the external part of the tooth, anti-bacterial treatment to slow down the biofilm recolonization or healing helping means with anti-inflammatory ingredients or even more bone growth factors.

For supragingival treatments such as fluoridization, creams or varnishes are available that can be deposited onto the teeth.

EP 2 455 064 A1 discloses polymer filled sheets like Periochip for subgingival treatments. In DE 10 114244 A1, thick gels as for example Ligosan for subgingival anti-bacterial maintaining are described.

However, all these methods have their limitations. For the supragingival deposition the contact time is often too short to get a real chemical effect. For example, the patient is usually not willing to remain for 20 minutes or longer on the dental chair to allow the chemical reaction to start. Further, the aqueous environment of the mouth quickly solves and removes all water-soluble compounds. For the subgingival additives, the presence of the support material which is quite thick is also a barrier against the soft tissue re-attachment. Furthermore, higher product concentrations are needed.

Therefore, it is an object of the present invention to provide a protection system for tooth surfaces that overcomes the above problems, which is easily applicable and can be used as delivery system for active ingredients such as pharmaceutically active compounds.

This object is achieved according to the invention by a powder for use in treating tooth surfaces with a powder jet device according to claim 1. The object is also achieved by the coating according to claim 14 and the use of the powder according to claim 15. Preferred embodiments of the invention are subject to the dependent claims as well as the following description and Figures.

One embodiment of the invention relates to a powder for use in treating tooth surfaces with a powder jet device, wherein the powder comprises at least one organic compound, wherein the at least one organic compound has a melting point <165 C and a solubility in water at 25° C. of <10 g/l.

It has surprisingly been found that the powder according to the invention, when applied to tooth surfaces with a powder jet device, gives a stable coating on the tooth surfaces which protects the tooth. Furthermore, if the powder additionally comprises an active ingredient, the coating can deliver the active ingredient over a defined period of time, for example for 20 minutes to several weeks. When the powder according to the invention is projected onto a surface with a stream of air, i.e. when using the powder in a powder jet device that is usually used to clean tooth surfaces, the powder will adhere to the tooth surface instead of abrading or cleaning the surface. Without being bound to this explanation, it is believed that due to the kinetic energy delivered to the powder, the powder undergoes a melting process at the point of collision with the tooth surface and forms a coating on the tooth surface (kinetic melting). This coating allows to protect the tooth surface.

If an active ingredient is additionally contained in the powder, it becomes part of the coating. The active ingredient (active compound), which is understood according to the invention as having an effect on the teeth or the human or animal body, can preferably be a pharmaceutically active compound, tooth fluoridization agent or tooth remineralization agent. Thereby, the invention allows to create an intelligent delivery system for an active ingredient, for example a pharmaceutically active compound, which is placed on the tooth surface and with a release of the active ingredient given by the solubility and/or biodegradability of the powder that functions as a support material for the active ingredient.

The active ingredient according to the invention is preferably selected from the group consisting of anti-inflammatory agents, anti-microbial agents, anti-bacterial agents, anti-viral agents, bones growth factors, tooth fluoridization agents and tooth remineralization agents. Anti-inflammatory agents, anti-microbial agents, anti-bacterial agents, anti-viral agents, bones growth factors are understood to be pharmaceutically active compounds or pharmaceutically active ingredients. The powder can comprise one or more active ingredients, for example one, two, three, four or five.

One advantage of the powder according to the invention is that the use in a conventional powder jet device gives a stable coating in the right position at the tooth surface. The powder may comprise an active ingredient, for example a pharmaceutically active compound or ingredient, which means a therapeutically efficient amount of a pharmaceutically active compound or ingredient. The term “treating tooth surface(s)” means in the sense of the present invention “applying to tooth surface(s)”. Treating or applying to tooth surface(s) is a typical use of a powder jet device, wherein a powder is sprayed with a carrier medium, for example air, onto tooth surfaces. In a preferred embodiment of the invention, the powder for use in treating tooth surfaces with a powder jet device is a powder for use in coating tooth surfaces with a powder jet device.

Preferably, a coating of tooth surface(s) is obtained when the deposition efficiency of the powder according to the invention, measured in % (defined as the powder mass sticking on the surface divided by powder mass projected towards the surface) is ≥1%, more preferably ≥5%.

The coating and thereby a preferably contained active ingredient, preferably a pharmaceutically active compound or a tooth fluoridization agent or tooth remineralization agent, can stay long enough on the tooth surface to act on the tooth surface or to deliver the active ingredient from this coating directly to the tooth surface, i.e. in the correct position. It can also deliver the active ingredient, for example a pharmaceutically active compound, into the body over a defined period of time.

The powder according to the present invention is easily applicable with conventional powder jet devices, in particular after tooth cleaning and the same powder jet device can be used for the cleaning and, subsequently, for applying the powder according to the invention to the cleaned tooth surfaces. Only the powder has to be changed.

In a preferred embodiment of the invention, the powder for use in treating tooth surfaces with a powder jet device comprises >20 wt.-%, based on the total weight of the powder, more preferably >25 wt.-%, even more preferably >50 wt.-%, even more preferably >70 wt.-% and most preferably >90 wt.-% of the at least one organic compound having a melting point <165° C. and a solubility in water at 25° C. of <10 g/l. At least one means according to the invention one or more, for example one, two, three, four or five. The one or more organic compounds preferably each have the defined characteristics, for example a melting point <165° C. and a solubility in water at 25° C. of <10 g/l.

Within the meaning of the present invention, it should be understood that the amounts of the components of the powder, given in wt.-% (weight-%), sum up to 100%.

An organic compound according to the invention is understood to be a chemical compound containing one or more carbon atom(s).

In a preferred embodiment of the invention, the melting point of the organic compound in the powder for use in treating tooth surfaces with a powder jet device is <162° C., more preferred <160° C., even more preferred <158° C. and most preferred <156° C. The low melting points give an advantageous coating as the kinetic melting is promoted. The solubility of the organic compound in water at 25° C. is preferably <5 g/l (gram per liter), more preferably <2 g/l and most preferably <1 g/l in water at 25° C. The solubility in water is related to distilled water (pH about 7.0). The limited solubility in water enhances the stability of the coating in the aqueous environment of the mouth.

In the course of the invention, it has been found that the organic compound of the powder according to the invention is preferably selected from the group consisting of fatty acids, fatty alcohols, sterols, hydroxy-substituted aromatic compounds and mixtures thereof. The fatty acids, fatty alcohols, sterols and hydroxy-substituted aromatic compounds can be optionally substituted and/or (partially) saturated when possible.

The fatty acids are preferably carboxylic acids with 10 to 24 carbon atoms, more preferably 12 to 22 carbon atoms, even more preferred are tridecanoic acid, pentadecanoic acid, palmitic acid, heptadecanoic acid, stearic acid, arachidic acid and mixtures thereof. The most preferred fatty acids are palmitic acid, stearic acid, arachidic acid and mixtures thereof.

The fatty alcohols are preferably alcohols with 10 to 24 caron atoms, more preferably 12 to 22 carbons, even more preferably stearyl alcohol or lauryl alcohol.

The sterol is preferably selected from the group consisting of cholesterol, beta-sistosterol, campesterol, stigmasterol, ergosterol, brassicasterol and mixtures thereof. The sterol is more preferably cholesterol and/or beta-sistosterol.

The hydroxy-substituted aromatic compounds are preferably hydroxy benzenes or a hydroxy toluenes, preferably butylated hydroxytoluene (BHT) or butylated hydroxy anisole (BHA). Most preferred as hydroxy-substituted aromatic compound is butylated hydroxytoluene (BHT).

The powder according to the invention is deposited on the tooth surface due to the kinetic energy delivered to the powder with the air. The deposition can be made supragingivally on enamel or exposed dentine, for example with the standard AIRFLOW handpiece of EMS or it can be made subgingivally, for example with the EMS PERIOFLOW subgingival nozzle. In a preferred embodiment of the invention, the powder should be projected towards the tooth surface with a powder jet device together with a gaseous carrier medium, in particular air. The powder is projected towards the tooth surface with the air. The air may contain water, preferably wherein water represents less than 10 mL/min, in particular, less than 5 mL/min. In a preferred embodiment, the powder jet device is used in the absence of water.

The use in treating tooth surfaces with a powder jet device according to the invention means spraying the powder onto a tooth surface with a conventional powder jet device. The powder then forms a coating on the tooth surface.

A powder according to the invention means a substance in the form of small particles. The average particle size (d₅₀) of the powder according to the invention is preferably 0,5 to 500 μm, more preferably 1-300 μm, even more preferably 2 to 200 μm, most preferably 5 to 100 μm. The particle sizes may be adapted to the field of application. For example, a smaller average particle size of the particles is preferred for treating subgingival tooth surfaces, in particular about 0,5 to 50 μm, more preferably 1 to 30 μm. For treating supragingival tooth surfaces, a larger average particle size can be used, preferably 0,5 to 500 μm, more preferably 2 to 200 μm.

In the context of this invention, the d₅₀ value is the particle size at which 50% of the particles are smaller than the d₅₀ value in terms of volume and 50% of the particles are larger than the d₅₀ value in terms of volume. This applies also to the d₁₀₀ values, the so-called maximum particle size. The d-values according to the invention are determined by laser diffraction method using a dry dispersion unit (Malvern Master-sizer 2000, equipped with a Scirocco dry dispersion unit, operated at 1.5 bar).

In a further preferred embodiment of the invention, the powder additionally comprises a flow aid, a bleaching agent and/or a flavouring agent. The total amount of these additional substances is preferably 0.2 to 5 wt.-%, based on the total weight of the powder, more preferably 0.5 to 2 wt.-%.

The flow aid is preferably selected from the group consisting of silicon dioxide (silica, in particular amorphous silica), aluminium silicate and/or aluminium hydroxide. Silicon dioxide is more preferred, in particular in an amount of 0.2 to 3 wt.-%, most preferably 0.5 to 2 wt.-%, based on the total weight of the powder.

Preferred bleaching agents are peroxides such as magnesium, calcium or zinc peroxides, persulfates such as sodium, potassium or ammonium persulfates or perborates.

In a preferred embodiment of the invention, the powder comprises less than 10 wt.-% of an abrasive powder (abrasive powder jet cleaning powder), more preferably less than 5 wt.-%, even more preferably less than 2 wt.-%, in particular less than 1 wt.-% of an abrasive powder, each based on the total weight of the powder. According to the invention, an abrasive powder or abrasive jet cleaning powder is meant to be a powder that is used in a powder jet device for cleaning tooth surfaces. The abrasive powders or abrasive jet cleaning powders are preferably sodium hydrogen carbonate, calcium carbonate, aluminium hydroxide, alditols, amino acids, sugars, cyclodextrins or mixtures thereof. Therefore, the powder according to the invention contains preferably less than 10 wt.-%, more preferably less than 5 wt.-%, even more preferably less than 2 wt.-%, most preferably less than 1 wt.-%, based on the total weight of the powder, of sodium hydrogen carbonate, calcium carbonate, aluminium hydroxide, alditols, amino acids, sugars, cyclodextrins or mixtures thereof. The alditol is preferably erythritol and the amino acid preferably glycine. The sugar is preferably trehalose or tagatose. Therefore, it is preferred that the powder according to the invention comprises less than 10 wt.-%, more preferably less than 5 wt.-%, even more preferably less than 2 wt.-%, most preferably less than 1 wt.-%, based on the total weight of the powder, of sodium hydrogen carbonate, calcium carbonate, aluminium hydroxide, erythritol, glycine, trehalose, tagatose, cyclodextrins or mixtures thereof.

In a further preferred embodiment of the invention, the powder comprises less than 10 wt.-%, preferably less than 5 wt.-%, more preferably less than 2 wt.-%, most preferred less than 1 wt.-%, based on the total weight of the powder, of one or more chemical compound(s) having a melting point >165° C., more preferably having a melting point >162° C., most preferably >160° C., and/or having a solubility in water at 25° C. of >10 g/l, preferably >5 g/l, more preferably >2 g/l, most preferably >1 g/l.

The powder according to the invention may comprise an active ingredient. An active ingredient is understood according to the invention as an active ingredient which may have an effect on the teeth or the human or animal body in general. Preferred active ingredients according to the invention are pharmaceutically active compounds, tooth fluoridization agents or tooth remineralization agents, more preferably anti-inflammatory agents, anti-microbials, anti-bacterial agents, anti-viral agents, bone growth factors, tooth fluoridization agents and tooth remineralization agents (tooth repair agents).

The anti-inflammatory agent according to the invention is preferably selected from the group consisting of Aceclofenac, Acemetacin, Acetaminosalol, Acetylsalicylic Acid, Alclofenac, Alminoprofen, α-Bisabolol, Amfenac, Bromfenac, Benoxaprofen, Benzpiperylon, Bermoprofen, Bromosaligenin, Bucloxic Acid, Bufexamac, Bumadizon, Butibufen, Carprofen, Cinmetacin, Clidanac, Clopirac, Diclofenac, Diclofenac Sodium, Diflunisal, Ditazol, Enfenamic Acid, ε-Acetamidocaproic Acid Bendazac, Etodolac, Etofenamate, Felbinac, Fenbufen, Fenclozic Acid, Fendosal, Fenoprofen, Fentiazac, Fepradinol, Flufenamic Acid, Flunoxaprofen, Flurbiprofen, Gentisic Acid, Glucametacin, Glycol Salicylate, Ibufenac, Ibuprofen, Ibuproxam, Indomethacin, Indoprofen, Isofezolac, Isoxepac, Isoxicam, Ketoprofen, Ketorolac, Lomoxicam, Lonazola, Lonazolac, Loxoprofen, Meclofenamic Acid, Meloxicam, Mesalamine, Metiazinic Acid, Mofebutazone, Mofezolac, Naproxen, Niflumic Acid, Olsalazine, Oxaceprol, Oxametacine, Oxaprozin, Oxicams, Oxyphenbutazone, Paranyline, Parsalmide, Perisoxal, Phenyl Salicylate, Pirazolac, Piroxicam, Pirprofen, Pranoprofen, Proprionic Acids, Protizinic Acid, Salacetamide, Salicilic Acid, Salicylamide O-Acetic Acid, Salicylsulfuric Acid, Sulfasalazine, Sulindac, Suprofen, Suxibuzone, Talniflumate, Tenoxicam, Terofenamate, Tiaprofenic Acid, Tiaramide, Tinoridine, Tolfenamic Acid, Tolmetin, Tropesin, Ximoprofen, Zaltoprofen, Zileuton and Zomepirac.

The antimicrobial agent is preferably selected from the group consisting of sulfonamides, phenolics, quaternary ammonium salts, cetylpyridinium chloride (CPC), chlorhexidine and salts thereof. Anti-bacterial agents are preferably penicillins, cephalosporins, tetracycline, doxycycline, chloramphenicol, and erythromycin.

The bone growth factor is preferably Emdogain® (enamel matrix derivative), BMP (bone morphogenetic protein) or calcium phosphate. The tooth fluoridization agent can be any fluoride containing or delivering compound such as fluoride salts, for example sodium fluoride or amino fluorides etc. The remineralization agent is preferably selected from the group consisting of bioglass, nano-hydroxyapatite, amorphous hydroxy apatite and calcium phosphate.

According to a preferred embodiment of the invention, the powder comprises 0.01 to ≤30 wt.-%, based on the total weight of the powder, of an active compound or ingredient. More preferably, the powder comprises 0.1 wt.-% to 20 wt.-%, based on the total weight of the powder, of an active ingredient, even more preferably 0.1 wt.-% to 10 wt.-%.

It is also preferred that the powder for use in treating tooth surfaces with a powder jet device according to the invention consists of the described components.

The invention also relates to a coating obtainable by applying the powder according to the invention to a surface, preferably to a tooth surface with a powder jet device. The coating preferably has a thickness of <100 μm, more preferably <50 μm, most preferably <20 μm.

The above thickness of the coating is the average thickness, determined by calculating the arithmetic average of 3 to 5 measurements of the thickness. The thickness is determined by optical measurement, for example 3D Microscope, step height measurement between a non-coated surface and coated surface (Keyence, VH-6000).

The invention also relates to the use of the powder according to the invention in treating tooth surfaces or applying the powder to tooth surfaces. The invention further relates to the use of the powder according to the invention in a powder jet device. This use comprises powder spraying, wherein the powder according to the invention is sprayed with a powder jet device onto the tooth surfaces together with a gaseous carrier medium, in particular air.

The invention further relates to a process of forming a coating on a tooth surface by applying the powder according to the invention to a tooth surface with a powder jet device. The invention also relates to coating on a tooth surface obtainable by applying the powder according to the invention to a tooth surface with a powder jet device.

The following examples provide preferred embodiments according to the invention and further exemplify the invention.

### Examples

The device used was an Handy 3.0 PERIO from EMS using a standard handpiece. Powder chamber was filled with a small amount of powder (about 0.2 g). The device was fixed on an x-y table in order to maintain fixed distance and angulation. Input pressure for the device was 3.8 bar (static). The device was operated at 2 mm from the surface and 90° angle. The deposition time was 40 seconds. The x-y table was a free moving table allowing the device to manually moved. During the 40 seconds, the device was therefore continuously slowly moved at about 0.4 mm/s with a Z movement in order to avoid double coverage of the powder. The deposition substrate was a microscope glass slide.

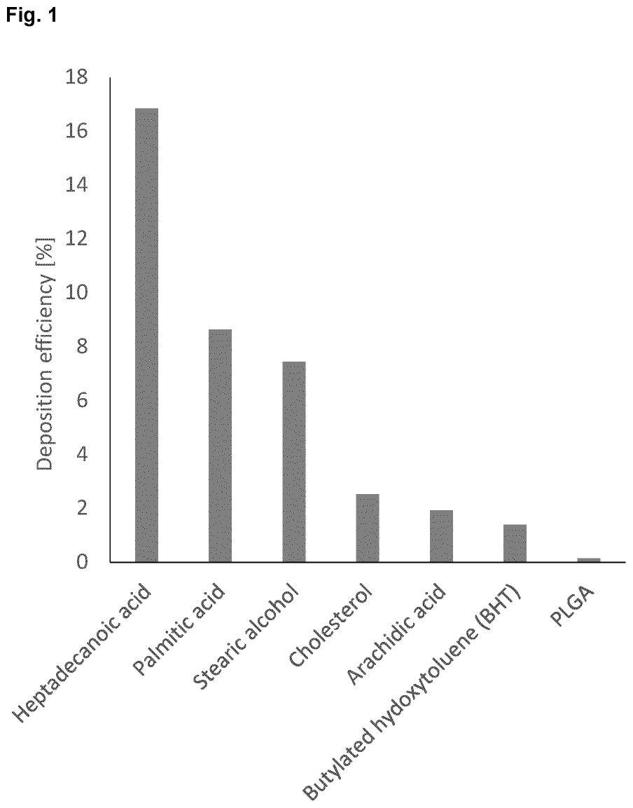

The reference material for the powder is PLGA (poly(lactid-co-glycolid)) which is a possible drug delivery material. The powder is formulated with a small amount of about 0.5-2 wt.-% of amorphous silica to improve powder fluidization. With regards to PLGA, different new materials have been tested according to FIG. 1.

FIG. 1 shows the deposition efficiency in % (powder mass sticking on the surface divided by powder mass projected towards the surface) for heptadecanoic acid, palmitic acid, stearic alcohol, cholesterol, arachidic acid, butylated hydroxytoluene (BHT) and PLGA (particle size <120 μm).

The deposition efficiency is the powder mass sticking on the tooth surface after using the powder according to the invention in a powder jet device, wherein the powder is projected towards the tooth surface, divided by the total powder mass projected towards the tooth surface. The deposition efficiency can, for example, be measured by determining the weight of the powder that is projected towards the tooth surface (x) and determining the weight of the powder that does not stick to the surface after the projection (y). The deposition efficiency is in this case (x-y): x. If, for example, one gram of powder is projected towards the tooth surface and 0.9 g of the powder does not stick to the tooth surface after using the powder in a powder jet device, i.e. projecting the powder towards the tooth surface, the deposition efficiency in this case is 10%.

In a preferred embodiment of the invention, the deposition efficiency of the powder according to the invention is ≥1%, preferably ≥2%, even more preferably ≥5%.

It has surprisingly been found that fatty acids, hydroxy-substituted aromatic compounds and sterols are particularly efficient regarding deposition efficiency, which is defined to be the quantity of a powder which sticks to the surface versus quantity of powder delivered from the device. Fatty acids, sterols and hydroxy-substituted aromatic compounds have a deposition efficiency which is ten times higher as the deposition efficiency of PLGA.

One of the reasons for this enhanced efficiency is considered to be the melting point of the fatty acids, sterols and hydroxy-substituted aromatic compounds according to the examples, because PLGA usually has a melting point of around 170-200° C., depending on the ratio of lactic and glycolid in the PLGA (95:5 has a melting point of about 173° C. and 10:90 of about 200° C.). A melting point of less than 165° C. of the powder according to the invention, however, gives a much more efficient coating and therefore a much more efficient deposition efficiency of the powder when using it in a powder jet device for treating tooth surfaces.

For drug release, the powder may contain active components, like anti-inflammatory (e.g. aspirine), bone growth factor (e.g. BMP, Emdogain®, Calcium phosphate), anti-microbials (e.g. CPC, Chlorhexidine, antibiotics), fluoridization agents (sodium fluoride, amino-fluoride etc.), tooth repair agents (nano-hap, amorphous hap, calcium phosphate) or desensitizing agent (arginine, potassium chloride). The concentration of the additive is usually between 0.1 to 30 wt.-%., based on the total weight of the powder.

1. A powder for use in treating tooth surfaces with a powder jet device, the powder comprising at least one organic compound, wherein the at least one organic compound has a melting point <165° C. and a solubility in water at 25° C. of <10 g/l.

2. The powder according to claim 1, wherein the melting point of the organic compound is <160° C. and/or the solubility of the organic compound in water at 25° C. is <2 g/l.

3. The powder according to claim 1, wherein the organic compound is selected from the group consisting of fatty acids, fatty alcohols, sterols, hydroxy-substituted aromatic compounds and mixtures thereof.

4. The powder according to claim 3, wherein the fatty acid is selected from the group consisting of tridecanoic acid, pentadecanoic acid, palmitic acid, heptadecanoic acid, stearic acid, arachidic acid and mixtures thereof, and/or the fatty alcohol is selected from the group consisting of stearyl alcohol and lauryl alcohol, and/or the sterol is selected from the group consisting of cholesterol, beta-sistosterol and mixtures thereof and/or the hydroxy-substituted aromatic compound is selected from the group consisting of butylated hydroxytoluene (BHT), butylated hydroxy anisole (BHA) and mixtures thereof.

5. The powder according to claim 1, wherein the powder further comprises an active ingredient.

6. The powder according to claim 5, wherein the powder comprises 0.1 wt.-% to ≤30 wt.-%, based on total weight of the powder, of the active ingredient.

7. The powder according to claim 5, wherein the active ingredient is selected from the group consisting of pharmaceutically active compounds, tooth fluoridation agents, tooth remineralization agents and mixtures thereof, preferably selected from the group consisting of anti-inflammatory agents, anti-microbial agents, anti-bacterial agents, anti-viral agents, bone growth factors, tooth fluoridation agents, tooth remineralization agents and mixtures thereof.

8. The powder according to claim 1, wherein the powder comprises less than 5 wt.-% of an abrasive powder.

9. The powder according to claim 1, wherein the powder comprises >20 wt.-%, based on total weight of the powder, of the at least one organic compound.

10. The powder according to any of claims 1 to 9, wherein the powder comprises >50 wt.-%, based on total weight of the powder, of the at least one organic compound.

11. The powder according to claim 8, wherein the abrasive powder is selected from the group consisting of sodium hydrogen carbonate, calcium carbonate, aluminium hydroxide, alditols, amino acids, sugars, cyclodextrins and mixtures thereof.

12. The powder according to claim 1, wherein the use in treating tooth surfaces with a powder jet device is a use in coating tooth surfaces with a powder jet device.

13. The powder according to claim 1, wherein the powder has a deposition efficiency of ≥1%, wherein the deposition efficiency is defined as the powder mass sticking on the tooth surface after using the powder in treating tooth surfaces with a powder jet device, wherein the powder is projected towards the tooth surface, divided by the powder mass projected towards the tooth surface.

14. A coating on a tooth surface obtainable by applying the powder according to claim 1 to a tooth surface with a powder jet device.

15. The powder according to claim 1 configured for use in a powder jet device.
